(19) United States
(12) Patent Application Publication
(10) Pub. No.: US 2025/0302940 A1
(43) Pub. Date: Oct. 2, 2025

(54) PATHOGEN-LIKE ANTIGEN-BASED VACCINE AND PREPARATION METHOD THEREFOR

(71) Applicants: RONGSEN BIOTECHNOLOGY (BEIJING) CO., LTD, Beijing (CN); INSTITUTE OF BIOPHYSICS, CHINESE ACADEMY OF SCIENCES, Beijing (CN)

(72) Inventors: Baidong HOU, Beijing (CN); Zhaolin HUA, Beijing (CN); Chang GUO, Beijing (CN)

(21) Appl. No.: 18/267,379

(22) PCT Filed: Dec. 15, 2021
(86) PCT No.: PCT/CN2021/138312

§ 371 Date: Jan. 8, 2024

(30) Foreign Application Priority Data
Dec. 15, 2020 (CN) 202011480939.4

(51) Int. Cl. A61K 39/215; A61K 39/00; A61K 39/145; A61K 39/385; A61P 31/14; C07K 14/005; C12N 7/00

(52) U.S. Cl. CPC A61K 39/215; A61K 39/001166; A61K 39/145; A61K 39/385; A61P 31/14; C07K 14/005; C12N 7/00; A61K 2039/53; A61K 2039/575; A61K 2039/6031; C07K 2319/20; C07K 2319/735; C12N 2710/12022; C12N 2710/12034; C12N 2760/16122; C12N 2760/16134; C12N 2770/20022; C12N 2770/20034; C12N 2795/18122; C12N 2795/18123; C12N 2795/18134

(57) ABSTRACT

The present application relates to a pathogen-like antigen (PLA) complex, and a preparation method therefor and an application thereof. The PLA complex consists of structurally-modified Escherichia coli bacteriophage virus-like particles (VLPs) and antigens displayed thereon, and nucleic acids are encapsulated inside the VLPs.

### SEQUENCE LISTING

The instant application contains a Sequence Listing which has been submitted electronically in ASCII format and is hereby incorporated by reference in its entirety. Said ASCII copy, created on Oct. 10, 2023, is named 51728-005001_Sequence_Listing_10_10_23_ST25 and is 19,279 bytes in size.

### TECHNICAL FIELD

The present invention relates to vaccines and immunotherapy techniques. Specifically, it involves a pathogen-like antigen (PLA) vaccine and the preparation method thereof, as well as the use of the vaccine for preventing or treating related diseases.

### BACKGROUND OF THE INVENTION

The subunit vaccine based on purified protein has the advantages of clear composition, good safety, and easy production, which is the direction of modern vaccine technology development. However, in application, protein subunit vaccines have also been found to have weak immunogenicity, making it difficult to generate high-level and long-lasting immune protection. At present, the commonly used method to improve the immunogenicity of subunit protein antigens is to add adjuvants. Adjuvants can enhance antigen specific immune responses by stimulating natural immune signals. However, overactivated natural immune signals may also produce unacceptable inflammatory side effects. This limits the effectiveness of using adjuvants to enhance the immunogenicity of protein subunit vaccines.

In subunit protein vaccines, some special recombinant proteins can be assembled into virus-like particles (VLP), such as the VLP antigen of hepatitis B virus surface antigen (HBsAg). The immunogenicity of these antigens will also be improved, which may be related to the increase in antigen density. However, this method is limited to proteins that can self assemble into VLPs. Moreover, the enhancement of immunogenicity by VLP alone is still limited, and adjuvants are still needed to achieve better results. A class of VLPs (including Qβ, MS2, AP205, etc.) from Escherichia coli phages have been found to have strong immunogenicity (Paul Pumpens et al., The True Story and Advantages of RNA Phage Capsids as Nanotools, Intervirology, 2016.11, 59:74-110). Thomas M. Kundig et al. (Thomas M. Kundig et al., Der p 1 peptide on virus-like particles is safeand highly immunogenic in healthy adults, J Allergy Clin Immunol, 2006, VOL. 117, NO. 6) found that antigens connected to Qβ-VLP surface through genetic engineering methods or chemical crosslinking methods can significantly enhance the specific immune response to antigens. However, the drawbacks of this approach are: 1) the limited variety of expressed fusion antigens by genetic engineering (antigens cannot be assembled into VLPs after fusion expression); 2) the method of chemical coupling is not suitable for large-scale production, and the orientation of antigens on the surface of VLP is not controllable, which affects immune efficacy.

AP205 protein (hereinafter referred to as AP205) is the main capsid protein of the newly identified AP205 RNA phage. In vitro, AP205 can self assemble into VLP particles, each containing 180 AP205 capsid protein molecules. The N-terminus and C-terminus of AP205 can be connected to the target protein. The SpyTag (ST)/SpyCatcher (SC) system originates from the CnaB2 domain and can spontaneously form stable isopeptide bonds under various conditions. Therefore, it can be used to solve the problem of protein subunit fusion expression and chemical coupling. However, we found that there is a significant mismatch between antigen proteins and VLP, which can easily lead to VLP aggregation and precipitation, seriously affecting the efficacy of the vaccine.

The present invention is committed to solving the afore-mentioned defects and deficiencies in the prior art.

### SUMMARY OF THE INVENTION

In order to address the shortcomings of the existing technology mentioned above, we have conducted a series of explorations and improvements on the protein engineering vaccine of pathogen-like antigen (PLA).

In one aspect, the present invention relates to a soluble pathogen-like antigen (PLA) complex comprising:
(1) a virus-like particle, which is self-assembled by a first fusion protein comprising a viral capsid protein or a variant thereof at its N-terminus, and SpyTag at its C-terminus; (2) a second fusion protein, comprising an antigen or a variant thereof, and SpyCatcher;
wherein the virus-like particle also encapsulates nucleic acid inside it, and wherein the antigen or a variant thereof is displayed on the surface of the virus-like particle through covalent connection between the SpyCatcher and the Spy Tag.

The soluble pathogen-like antigen complex of the present invention, wherein the nucleic acid encapsulated in the virus-like particle is the nucleic acid which is encapsulated by the virus-like particle when self-assembling and is from host bacteria used to express the virus-like particles, preferably the host bacteria is Escherichia coli, and preferably the nucleic acid is RNA.

The soluble pathogen-like antigen complex of the present invention, wherein the capsid protein is from Escherichia coli phage Qβ, MS2 or AP205.

The soluble pathogen-like antigen complex of the present invention, wherein the capsid protein is from Escherichia coli phage AP205.

The soluble pathogen-like antigen complex of the present invention, wherein the antigen is selected from RBD sequence of S protein of SARS-CoV2 virus, African swine fever virus antigen eP22, influenza virus antigen M2E and autoantigen myelin oligodendrocyte glycoprotein MOG.

The soluble pathogen-like antigen complex of the present invention, wherein the sequence of the capsid protein of the phage AP205 has at least 90%, 91%, 92%, 93%, 94%, 95%, 96%, 97%, 98%, 99% or 100% identity with SEQ ID NO: 1.

The soluble pathogen-like antigen complex of the present invention, wherein the sequence of the capsid protein of the phage AP205 is SEQ ID NO: 1.

The soluble pathogen-like antigen complex of the present invention, wherein the sequence of the SpyTag has at least 90%, 91%, 92%, 93%, 94%, 95%, 96%, 97%, 98%, 99% or 100% identity with of SEQ ID NO: 3, wherein the sequence of the SpyCatcher has at least 90%, 91%, 92%, 93%, 94%, 95%, 96%, 97%, 98%, 99% or 100% identity with SEQ ID NO: 4.

The soluble pathogen-like antigen complex of the present invention, wherein the sequence of the SpyTag is SEQ ID NO: 3, the sequence of the SpyCatcher is SEQ ID NO: 4.

The soluble pathogen-like antigen complex of the present invention, wherein an isopeptide bond is formed between Asp at position 7 of the SpyTag sequence SEQ ID NO: 3 and Lys at position 31 of the SpyCatcher sequence SEQ ID NO: 4.

The soluble pathogen-like antigen complex of the present invention, wherein in the first fusion protein, the phage capsid protein or a variant thereof is linked with the SpyTag via a first linker peptide, and in the second fusion protein, the antigen or a variant thereof is linked to the SpyCatcher via a second linker peptide.

The soluble pathogen-like antigen complex of the present invention, wherein the sequence of the first linker peptide is SEQ ID NO: 5, the sequence of the second linker peptide is SEQ ID NO: 6.

The soluble pathogen-like antigen complex of the present invention, wherein the second fusion protein is connected to the virus-like particle at a ratio of less than or equal to 1:1 according to different antigents, preferably connected at a ratio of 1:1, 1:1.5, 1:2, 1:3, 1:4, 1:5, 1:6, 1:7, 1:8, 1:9, 1:10, 1:11, 1:12, so as to ensure the solubility and immunogenicity of the pathogen-like antigen complex, the ratio is calculated as the ratio of the SpyCatcher on the second fusion protein to the SpyTag on the virus-like particle.

The pathogen-like antigen vaccine composition of the present invention, formulated as a vaccine composition, together with pharmaceutically acceptable carriers and/or excipients.

In another aspect, the present invention provides a method for preparing a soluble pathogen-like antigen complex, comprising purifying the virus-like particles at a pH range of 4.0 to 9.0, preferably 5.5 to 8.5.

In another aspect, the present invention provides a method for improving the solubility of pathogen-like antigen complexe, comprising the following steps: (1) preparing the virus-like particle and the second fusion protein as defined above; and (2) when connecting the second fusion protein with the virus-like particle, reducing a connection ratio between the second fusion protein and the virus-like particle to obtain a soluble pathogen-like antigen complex.

The method for improving the solubility of pathogen-like antigen complexe of the present invention, wherein the antigen is selected from RBD sequence of S protein of SARS-CoV2 virus, African swine fever virus antigen eP22, influenza virus antigen M2E or autoantigen myelin oligodendrocyte glycoprotein MOG.

In a further aspect, the present inentin relates to a method for preventing and/or treating diseases associated with SARS-CoV2 virus, influenza virus, African swine fever virus or autoantigen myelin oligodendrocyte glycoprotein MOG in a subject in need thereof, comprising administering a preventive and/or therapeutically effective amount of the soluble pathogen-like antigen complex or vaccine composition of the present invention to the subject.

The present invention has the following advantages:
1) It effectively avoids particle aggregation or precipitation caused by poor compatibility between antigen proteins and linkers, or between antigen proteins and VLPs, which facilitates vaccine production and ensures stable vaccine efficacy.
3) The method of preparing a pathogen-like antigen vaccine of the present invention avoids the degradation of nucleic acid during the separation and purification process of PLA vaccines, enabling effective improvement of the immunogenicity of the vaccine without addition of adjuvants, while also reducing or avoiding excessive inflammatory reactions caused by the addition of adjuvants.

### BRIEF DESCRIPTION OF THE DRAWINGS

FIG. 1: Lysis of whole bacteria before and after induced expression of AP205 fusion protein, wherein FIG. 1A is SC-AP205 and FIG. 1B is AP205-SC.

FIG. 2: Nucleic acid gel image of SC-AP205 and AP205-SC after sucrose cushion centrifugation, wherein 1 is AP205-ST, 2 is SC-AP205, and 3 is AP205-SC.

FIG. 3: Gel image of layered collection protein of AP205 fusion protein after cesium chloride density gradient centrifugation, wherein FIG. 3A is SC-AP205, and FIG. 3B is AP205-SC.

FIG. 4: Whole bacterial lysis before and after AP205-ST induction.

FIG. 5: Gel image of layered collection protein of AP205-ST after cesium chloride density gradient centrifugation.

FIG. 6: Degradation of RBD-SC.

FIG. 7: Degradation of AP205-RBD (SC at C-terminus).

FIG. 8: Stability of the ligated product of SC-RBD and AP205-ST.

FIG. 9: The effect of the modified AP205 on the solubility of the connected product, \textcircled{1} is the wild-type AP205, and \textcircled{2} is the modified AP205 of the present invention. FIG. 9A and FIG. 9B are SDS-PAGE and nucleic acid gel images, respectively.

FIG. 10: The effect of adjusting the antigen ratio on the solubility of the connected product, FIG. 10A, FIG. 10B and FIG. 10C are SDS-PAGE, nucleic acid gel, and Coomassie R-250 images, respectively.

FIG. 11: Connection between African swine fever antigen eP22 and AP205-ST. FIGS. 11A, 11B, and 11C show SDS-PAGE, nucleic acid gel, and Coomassie R-250 images, respectively.

FIG. 12: Connection between influenza virus antigen M2E and AP205-ST. FIGS. 12A, 12B, and 12C show SDS-PAGE, nucleic acid gel, and Coomassie R-250 images, respectively.

FIG. 13: Connection between autoantigen MOG and AP205-ST. FIGS. 13A, 13B, and 13C show SDS-PAGE, nucleic acid gel, and Coomassie R-250 images, respectively.

FIG. 14: The effect of VLP purification condition on the presence of the nucleic acid inside the VLP. FIG. 14A and FIG. 14B are SDS-PAGE and nucleic acid gel images, respectively.

FIG. 15: Changes of nucleic acids inside VLPs under different pH gradients.

FIG. 16: Anti-RBD IgG antibodies produced in mice immunized with PLA-SARS-CoV2 vaccine (primary immunization).

FIG. 17: Anti-RBD IgG antibodies produced in mice immunized with PLA-SARS-CoV2 vaccine (secondary immunization).

FIG. 18: Changes in titers of RBD IgG antibodies produced by PLA-SARS-CoV2 vaccine after primary immunization and re-immunization.

FIG. 19: The vaccine complex constructed by the VLP of the present invention and several other antigens has significantly enhanced the ability to induce antibodies compared with the traditional vaccine added with adjuvant (FIG. 19A, FIG. 19B and FIG. 19C use African swine fever virus antigen eP22, influenza virus antigen M2E and autoantigen myelin oligodendrocyte glycoprotein MOG, respectively).

FIG. 20: Neutralizing antibodies produced in mice immunized with PLA-SARS-CoV2 vaccine.

FIG. 21: Anti-RBD IgG antibodies produced in macaques immunized with PLA-SARS-CoV2 vaccine.

FIG. 22: Neutralizing antibodies produced in macaques immunized with PLA-SARS-CoV2 vaccine.

FIG. 23: Viral load in the lung of macaques immunized with PLA-SARS-CoV2 vaccine.

### DETAILED DESCRIPTION OF THE INVENTION

To address the issue of VLP aggregation and precipitation caused by the imcompatibility between antigen proteins and VLP, which affects the stability and immune efficacy of vaccines, the inventor has conducted extensive research.

The inventor accidentally discovered that the modification of the phage AP205 capsid protein sequence (hereinafter referred to as AP205 sequence) assembled into virus-like particles can significantly improve the solubility of the connected vaccine product (Example 3). The ratio of the two when the chassis particle is connected to the antigen would also affect the solubility of the connected product (Example 4). The inventor also unexpectedly found that the fusion protein constructed by AP205 sequence and the SpyCatcher sequence cannot be normally assembled into a virus-like particle, and only the fusion protein formed by constructing Spy Tag at the C-terminus of the AP205 sequence can be assembled normally (Example 1). Although the SpyCatcher sequence can be located at the N-terminus or C-terminus of the antigen sequence, the fusion protein formed when SpyCatcher is located at the N-terminus of the antigen is relatively more stable, and the connected product formed by connecting the fusion protein with virus-like particle carrying the Spy Tag sequence is also more stable (Example 2).

The aggregation or precipitation of pathogen-like antigen complexes would directly affect the vaccine's ability to regulate specialized antigen presenting function of B cells, seriously affecting vaccine production and stability of efficacy. Therefore, ensuring the solubility of particles obtained after connecting antigen and VLP is a key factor for PLA vaccines to play their due role.

The RNA nucleic acid inside VLP is another key factor in the effectiveness of adjuvant-free PLA vaccines. During the separation and purification of PLA vaccines, although certain conditions do not affect the stability of the protein, they may cause RNA degradation. The inventor's research found that a high pH value in the solution during the VLP purification process can damage or even completely degrade the RNA in the VLP. An appropriate pH value can ensure the retention of RNA components within the VLP.

In one aspect, the present invention relates to a soluble pathogen-like antigen complex comprising:
(1) a virus-like particle, which is self-assembled by a first fusion protein comprising a viral capsid protein or a variant thereof at its N-terminus, and SpyTag at its C-terminus; (2) a second fusion protein, comprising an antigen or a variant thereof, and SpyCatcher;
wherein the virus-like particle also encapsulates nucleic acid inside it, and wherein the virus-like particle and the second fusion protein are covalently connected through the SpyCatcher and the SpyTag, so that the antigen or a variant thereof is displayed on the surface of the virus-like particle.

The soluble pathogen-like antigen complex of the present invention, wherein the nucleic acid encapsulated in the virus-like particle is the nucleic acid from host bacteria used to express the virus-like particles, preferably the host bacteria is Escherichia coli, and preferably the nucleic acid is RNA.

The soluble pathogen-like antigen complex of the present invention, wherein the capsid protein is from Escherichia coli phage Qβ, MS2 or AP205.

The soluble pathogen-like antigen complex of the present invention, wherein the capsid protein is from Escherichia coli phage AP205.

The soluble pathogen-like antigen complex of the present invention, wherein the antigen is selected from RBD sequence of S protein of SARS-CoV2 virus, African swine fever virus antigen eP22, influenza virus antigen M2E and autoantigen myelin oligodendrocyte glycoprotein MOG.

The soluble pathogen-like antigen complex of the present invention, wherein the sequence of the capsid protein of the phage AP205 has at least 90%, 91%, 92%, 93%, 94%, 95%, 96%, 97%, 98%, 99% or 100% identity with SEQ ID NO: 1.

The soluble pathogen-like antigen complex of the present invention, wherein the sequence of the capsid protein of the phage AP205 is SEQ ID NO: 1.

The soluble pathogen-like antigen complex of the present invention, wherein the sequence of the SpyTag has at least 90%, 91%, 92%, 93%, 94%, 95%, 96%, 97%, 98%, 99% or 100% identity with of SEQ ID NO: 3, wherein the sequence of the SpyCatcher has at least 90%, 91%, 92%, 93%, 94%, 95%, 96%, 97%, 98%, 99% or 100% identity with SEQ ID NO: 4.

The soluble pathogen-like antigen complex of the present invention, wherein the sequence of the SpyTag is SEQ ID NO: 3, the sequence of the SpyCatcher is SEQ ID NO: 4.

The soluble pathogen-like antigen complex of the present invention, wherein an isopeptide bond is formed between Asp at position 7 of the SpyTag sequence SEQ ID NO: 3 and Lys at position 31 of the SpyCatcher sequence SEQ ID NO: 4.

The soluble pathogen-like antigen complex of the present invention, wherein in the first fusion protein, the phage capsid protein or a variant thereof is linked with the SpyTag via a first linker peptide, and in the second fusion protein, the antigen or a variant thereof is linked to the SpyCatcher via a second linker peptide.

The soluble pathogen-like antigen complex of the present invention, wherein the sequence of the first linker peptide is SEQ ID NO: 5, the sequence of the second linker peptide is SEQ ID NO: 6.

The soluble pathogen-like antigen complex of the present invention, wherein the second fusion protein is connected to the virus-like particle at a ratio of less than or equal to 1:1 according to different antigents, preferably connected at a ratio of 1:1, 1:1.5, 1:2, 1:3, 1:4, 1:5, 1:6, 1:7, 1:8, 1:9, 1:10, 1:11, 1:12, so as to ensure the solubility and immunogenicity of the pathogen-like antigen complex, the ratio is calculated as the ratio of the SpyCatcher on the second fusion protein to the SpyTag on the virus-like particle.

The present invention also relates to a nucleic acid sequence encoding the first fusion protein and the second fusion protein and a vector comprising the nucleic acid sequence.

In some embodiments, the nucleic acid sequence or nucleic acid molecule or vector described herein can be codon optimized.

In some embodiments, the nucleic acid sequence or nucleic acid molecule or vector described herein may be a degenerate version thereof.

The pathogen-like antigen vaccine composition of the present invention, formulated as a vaccine composition, together with pharmaceutically acceptable carriers and/or excipients.

In another aspect, the present invention provides a method for preparing a soluble pathogen-like antigen complex, comprising purifying the virus-like particles at a pH range of 4.0 to 9.0, preferably 5.5 to 8.5.

In another aspect, the present invention provides a method for improving the solubility of pathogen-like antigen complexe, comprising the following steps: (1) preparing the second fusion protein and the virus-like particle as defined in any one of preceding items; and (2) when connecting the second fusion protein with the virus-like particle, reducing a connection ratio between the second fusion protein and the virus-like particle to obtain a soluble pathogen-like antigen complex.

The method for improving the solubility of pathogen-like antigen complexe of the present invention, wherein the antigen is selected from RBD sequence of S protein of SARS-CoV2 virus, African swine fever virus antigen eP22, influenza virus antigen M2E or autoantigen myelin oligodendrocyte glycoprotein MOG.

In a further aspect, the present invention relates to a method for preventing and/or treating diseases associated with SARS-CoV2 virus, influenza virus, African swine fever virus or autoantigen myelin oligodendrocyte glycoprotein MOG in a subject in need thereof, comprising administering a preventive and/or therapeutically effective amount of the soluble pathogen-like antigen complex or the vaccine composition of the present invention to the subject.

In some embodiments, the associated diseases could be caused by the SARS-COV-2 virus and/or its mutants.

In some embodiments, the associated disease could be COVID-19.

The term “fusion protein” used herein refers to a genetically engineered protein encoded by a nucleotide sequence formed by two or more complete or partial genes or a series of nucleic acids combined together. Alternatively, fusion proteins can be made by combining two or more heterologous peptides.

The term “linker peptide” or “linker sequence” as used herein means one or more (e.g., about 2-10) amino acid residues between two adjacent motifs, regions or domains of a polypeptide, such as between the antigenic peptide or between the antigenic peptide and an adjacent peptide encoded by the multiple translation leader sequence, or between the antigenic peptide and a spacer or cleavage site. Linker peptides can be derived from the construct design of the fusion protein (e.g., amino acid residues resulting from the use of restriction enzyme sites during construction of a nucleic acid molecule encoding the fusion protein).

The term “variant” as used herein refers to a protein or nucleic acid molecule whose sequence is similar but not identical to a reference sequence, wherein the activity of the variant protein (or the protein encoded by the variant nucleic acid molecule) is not significantly altered. These variations in sequence may be naturally occurring variations or may be engineered using genetic engineering techniques known to those skilled in the art. Examples of such techniques can be found in Sambrook J, Fritsch E F, Maniatis T et al., in Molecular Cloning-A Laboratory Manual, 2nd Edition, Cold Spring Harbor Laboratory Press, 1989, pp. 9.31-9.57), or Current Protocols in Molecular Biology, John Wiley & Sons, N.Y. (1989), 6.3.1-6.3.6. With regard to variants, any type of change in amino acid or nucleic acid sequence is permissible as long as the activity of the resulting variant protein or polynucleotide is not significantly altered. Examples of such variations include, but are not limited to, deletions, insertions, substitutions, and combinations thereof. According to their properties, amino acids can be classified into charged amino acids, uncharged amino acids, polar uncharged amino acids, and hydrophobic amino acids. Thus, protein variants containing substitutions may be those in which amino acids are substituted by amino acids from the same group. Such substitutions are known as “conservative” substitutions.

As used herein, the term “antigen” or variants thereof means a polypeptide that can stimulate a cell to generate an immune response.

As used herein, the term “virus-like particles (VLPs)” is a particle assembled from one or more viral structural proteins, which has a similar external structure and antigenicity to viral particles, but does not contain viral genes.

The terms “vaccine” and “vaccine composition” used in the present invention refer to a pharmaceutical composition containing corresponding virus antigens, which can induce, stimulate or enhance the subject's immune response against the corresponding virus.

The term “nucleic acid” or “nucleic acid molecule” as used herein means any one of deoxyribonucleic acid (DNA), ribonucleic acid (RNA), oligonucleotides, fragments produced, for example, by polymerase chain reaction (PCR) or by in vitro translation, and fragments produced by any one or more of ligation, cleavage, endonuclease action, or exonuclease action. In certain embodiments, nucleic acids of the disclosure are produced by PCR. Nucleic acids can be composed of monomers that are naturally occurring nucleotides (such as deoxyribonucleotides and ribonucleotides), analogs of naturally occurring nucleotides (e.g., naturally occurring nucleotides in α-enantiomeric forms) or combinations thereof. Modified nucleotides may have modifications in or in place of sugar moieties, or pyrimidine or purine base moieties.

The term “construct” as used herein means any polynucleotide comprising a recombinant nucleic acid. The construct can be present in a vector (e.g., bacterial vector, viral vector), or can be integrated into the genome. A “vector” is a nucleic acid molecule capable of transporting another nucleic acid. A vector can be, for example, a plasmid, cosmid, virus, RNA vector or linear or circular DNA or RNA molecule, which can include chromosomal, non-chromosomal, semi-synthetic or synthetic nucleic acids. Exemplary vectors are those capable of autonomous replication (episomal vectors) and/or expression of nucleic acids to which they are linked (expression vectors).

As used herein, the terms “signal peptide” and “leader sequence” are used interchangeably herein and refer to an amino acid sequence that can be linked to the amino terminus of the proteins set forth herein. A signal peptide/leader sequence usually directs the localization of the protein. The signal peptide/leader sequence used herein preferably facilitates secretion of the protein from the cell in which it is produced. The signal peptide/leader sequence is often cleaved from the rest of the protein (often called the mature protein) after secretion from the cell. A signal peptide/leader sequence is attached to the N-terminus of the protein and is about 9 to 200 nucleotides (3 to 60 nucleic acids) in length. The signal peptide used in the present invention can be the signal peptide sequence of S protein of SARS-COV-2 virus or the signal peptide sequence from other eukaryotic/viral proteins.

The term “expression vector” as used herein refers to a DNA construct comprising a nucleic acid molecule operably linked to suitable control sequences enabling expression of the nucleic acid molecule in a suitable host. Such control sequences include a promoter for effecting transcription, optionally an operator sequence for controlling such transcription, a sequence encoding a suitable mRNA ribosomal binding site, and sequences controlling termination of transcription and translation. The vector can be a plasmid, phage particle, virus, or simply a potential genomic insert. Viral vectors can be DNA-based (eg, adenovirus or vaccinia virus) or RNA-based, including oncolytic viral vectors (eg, VSV), replication competent or non-replicative. Once transformed into a suitable host, the vector can replicate and function independently of the host genome, or, in some cases, can integrate into the genome itself. In this specification, “plasmid”, “expression plasmid”, and “vector” are often used interchangeably.

The term “expression” as used herein means the process of producing a polypeptide based on the nucleic acid sequence of a gene. The processes include transcription and translation. Translation can start at an unconventional start codon, such as the CUG codon, or translation can start at several start codons (standard AUG and unconventional) to produce more protein than mRNA produced (on a per mole basis quantity).

As used herein, the term “introducing” in the context of inserting a nucleic acid sequence into a cell means “transfection” or “transformation” or “transduction” and includes the integration of a nucleic acid sequence into a eukaryotic or prokaryotic cell, wherein the nucleic acid sequence can be integrated into the genome of the cell (e.g., chromosomal, plasmid, plastid, or mitochondrial DNA), transformed into an autonomous replicon, or expressed transiently (eg, transfected mRNA).

Recombinant methods for expressing exogenous or heterologous nucleic acids in cells are well known in the art. Such methods can be found, for example, in Sambrook et al., Molecular Cloning: A Laboratory Manual, Third Edition, Cold Spring Harbor Laboratory, New York (2001); and Ausubel et al., Current Protocols in Molecular Biology, John Wiley and Sons, Baltimore, MD (1999). Genetic modification of a nucleic acid molecule encoding a fusion antigen protein can confer biochemical or metabolic capabilities on a recombinant or non-natural cell altered from its naturally occurring state.

The term “host” as used herein refers to any organism or cell thereof into which the construct of the invention can be introduced, whether eukaryotic or prokaryotic, in particular, a host in which RNA silencing occurs. In specific embodiments, “hosts” include Escherichia coli such as E. coli. The term “host” refers to eukaryotes, including unicellular eukaryotes such as yeast and fungi and multicellular eukaryotes such as animals. Non-limiting examples include invertebrates (e.g., insects, coelenterates, echinoderms, nematodes etc.); eukaryotic parasites (e.g., malaria parasites such as Plasmodium falciparum, worms, etc.); vertebrates (e.g., fish, amphibians, reptiles, birds, mammals); and mammals (e.g., rodents, primates such as humans and non-human primates). Thus, the term “host cell” properly encompasses cells of such eukaryotes as well as cell lines derived from such eukaryotes.

The term “adjuvant” used herein refers to a natural or synthetic substance that promotes the body's T cell or B cell response by enhancing the activity of macrophages and participates in the immune response to haptens or antigens.

The term “prevention and/or treatment” as used herein refers to inhibiting the replication, spread or colonization of the corresponding virus in the host, as well as alleviating the symptoms of the virus-infected disease or disorder. The treatment is considered to be therapeutically effective if there is a reduction in viral load, a reduction in symptoms and/or an increase in food intake and/or growth.

“Therapeutically effective amount (or dose)” or “effective amount (or dose)” of a compound or composition as used herein means the amount of the compound sufficient to cause improvement of one or more symptoms of the disease being treated in a statistically significant manner. The precise amount depends on numerous factors, e.g., the activity of the composition, the method of delivery employed, the immunostimulatory capacity of the composition, the intended patient and patient considerations, etc., and can be readily determined by one of ordinary skill in the art. A therapeutic effect may include, directly or indirectly, the alleviation of one or more symptoms of a disease, and a therapeutic effect may also include, directly or indirectly, the stimulation of a cellular immune response.

As used herein, the term “pharmaceutically acceptable carrier” includes any carrier that does not, by itself, induce the production of antibodies deleterious to the individual receiving the pharmaceutical composition. Suitable carriers are usually large, slowly metabolized macromolecules, such as proteins, polysaccharides, polylactic acid, polyglycolic acid, amino acid polymers, amino acid copolymers, lipid aggregates (such as oil droplets or liposomes), and the like. These pharmaceutically acceptable carriers are well known to those of ordinary skill in the art.

The term “subject” as used herein may be any organism capable of mounting a cellular immune response, such as a human, pet, livestock, exhibit animal, zoo specimen, or other animal. For example, a subject can be a human, non-human primate, dog, cat, rabbit, rat, mouse, guinea pig, horse, cow, sheep, goat, pig, and the like. Subjects who need to be administered a therapeutic agent as described herein include those who have been infected by SARS-COV-2 virus or even have developed a virus infection-related disease, or are at risk of SARS-COV-2 virus infection.

The term “subject in need” as used herein means a subject at high risk of or suffering from a disease, disorder or condition. The disease, disorder, or condition is suitable for treatment or improvement with the compound or combination provided herein. In some embodiments, the subject in need is a human.

For complexes comprising pathogen-like antigens as described herein, the desired outcome is a safe product capable of inducing durable protective immunity with minimal side effects. Compared to other strategies (e.g., whole live or attenuated pathogens), being inexpensive to produce could minimize or eliminate contraindications that have otherwise been (generally) associated with the use of whole or attenuated viral immune compositions. The ability to quickly respond to infectious disease emergencies (natural outbreaks, large-scale epidemics, or bioterrorism) is a benefit of the effective application of the embodiments disclosed herein, whether in the context of biodefense, immunotherapy, or technology.

The pathogen-like antigen vaccines of the present invention can be administered by, for example, intramuscular injection, subcutaneously, intranasally, transmucosally presented, intravenously or by intradermal or subcutaneous administration.

The following will provide examples of the specific embodiments for the invention of pathogenic antigen vaccines. However, it should be understood that these examples do not in any way limit the scope of the present invention.

### EXAMPLES

The soluble pathogen-like antigen (PLA) vaccine of the present invention comprises four structural elements: 1) an antigen display chassis based on phage VLP or other nanoparticles; 2) The TLR stimulators carried within the chassis particles, such as nucleic acids, such as RNA, preferably from the expressing host; 3) The Sphere/Spytag sequence used to connect chassis particles with antigens; and 4) antigens to be displayed.

For the fusion protein containing the above four structural elements, the necessary conditions for it to be used as an adjuvant free protein engineering vaccine include that, the fusion protein has sufficient structural stability, is soluble, and would not accumulate or precipitate; meanwhile, the TLR stimulators encapsulated inside the chassis particles, such as nucleic acids, have not been degraded and eliminated. The inventor found that multiple factors affect the stability and solubility of fusion proteins used as protein engineering vaccines.

### Example 1: The Effect of the Linking Format of SpyCatcher (SC) and SpyTag (ST) with AP205 on VLP Self-Assembly

### (1) Construction of Fusion Protein SC-AP205 (SC is Located at the N-Terminus of AP205) Expression Plasmid

The amino acid sequence of SC is SEQ ID NO: 4. The amino acid sequence of modified AP205 (non-wild type) is SEQ ID NO: 1. The two are linked by a linker sequence SEQ ID NO: 5.

### I. Construction of AP205 Expression Vector

A full-length 393 bp cDNA (SEQ ID NO: 9) fragment encoding AP205 was artificially synthesized. A BamHI restriction site was added at its 5′ end and a GSGGSG linker, an AgeI restriction site, a stop codon TAA, and a KpnI restriction site were added at its 3′ end. The synthesized AP205 cDNA fragment (1 μg) and pET21 plasmid (1 μg) were digested with BamHI (Takara 1010A) and KpnI endonuclease (Takara 1068A) at 37° C. for 2 hours, respectively. The digested cDNA fragment and pET21a plasmid fragment were then separated by agarose gel electrophoresis. The isolated cDNA fragment and pET21a plasmid fragment were purified using a small amount of DNA product purification kit (Zhuangmeng Bio ZP201-3). The purified cDNA fragment was further subjected to DNA ligation reaction with the pET21a plasmid fragment to construct a pET21a plasmid containing the cDNA fragment (called pET21a-AP205 plasmid). The ligase was T4 DNA ligase (Takara 2011A), the ligation buffer was T4 DNA Ligase Buffer (Takara 2011A), the ratio of the pET21a plasmid fragment to the AP205 cDNA fragment in the ligation reaction was about 1:3, and the total DNA was about 200 ng. Ligation was done at 22° C. for 2 hours. The pET21a-AP205 plasmid was transformed into the expression host as follows: 15 μl of the ligation reaction solution was added to 150 μl of XLI-Blue competent E. coli (Transgene CD401-02) at 42° C. for 1 minute. 150 μl was pipetted onto an ampicillin-resistant LB plate and the plated was incubated at 37° C. for 14-16 hours. A single colony was taken from the plate, and the plasmid DNA was extracted with a plasmid purification kit (Transgene EM101-02) and verified by enzyme digestion, confirming the successful construction of the pET21a-AP205 plasmid.

### II. Construction of SC-AP205 Expression Vector

A full-length 276 bp cDNA fragment encoding SC (SEQ ID NO: 10) was artificially synthesized by PCR (upstream primer (SEQ ID NO: 13): acgggatccATGTCGTACTACCATCACCATC, downstream primer (SEQ ID NO: 14): cccggatccactgccgctacctccAATATGAGCGTCACCTTTAGTTGC, the PCR program is \textcircled{1} 94° C. for 5 minutes \textcircled{2} 94° C. for 30 seconds \textcircled{3} 58° C. for 30 seconds \textcircled{4} 72° C. for 1 minute, \textcircled{2} \textcircled{3} \textcircled{4} for 30 cycles, \textcircled{5} 72° C. for 5 minutes \textcircled{6} keeping at 4° C.). BamHI restriction sites were added at both its 5′ and 3′ ends. The synthesized SC cDNA fragment (1 μg) and pET21a-AP205 plasmid (1 μg) were digested with BamHI endonuclease (Takara 1010A) at 37° C. for 2 hours, respectively. The digested cDNA fragment and pET21a-AP205 plasmid fragment were then separated by agarose gel electrophoresis. The isolated cDNA fragment and the pET21a-AP205 plasmid fragment were purified using a small DNA product purification kit (Zhuangmeng Bio ZP201-3). The purified cDNA fragment was further subjected to DNA ligation reaction with the pET21a-AP205 plasmid fragment to construct the pET21a-AP205 plasmid containing the cDNA fragment (called pET21a-SC-AP205 plasmid). The ligase was T4 DNA ligase (Takara 2011A), the ligation buffer was T4 DNA Ligase Buffer (Takara 2011A), the ratio of the pET21a-AP205 plasmid fragment to the SC cDNA fragment in the ligation reaction was about 1:3, and the total DNA was about 200 ng. Ligation was done at 22° C. for 2 hours. The pET21a-SC-AP205 plasmid was transformed into the expression host as follows: 15 μl of the ligation reaction solution was added to 150 μl of XLI-Blue competent E. coli (Transgene CD401-02) at 42° C. for 1 minute. 150 μl was pipetted onto an ampicillin-resistant LB plate and the plate was incubated at 37° C. for 14-16 hours. A single colony was taken from the plate, and the plasmid DNA was extracted with a plasmid purification kit (Transgene EM101-02) and verified by enzyme digestion, confirming the successful construction of the pET21a-SC-AP205 plasmid.

### (2) Construction of Fusion Protein AP205-SC (SC is Located at the C-Terminus of AP205) Expression Plasmid

The fusion protein AP205-SC expression plasmid was prepared in the same way as in above (1), except that a full-length 276 bp SC cDNA fragment was artificially synthesized by PCR (upstream primer:
acgaccggtATGTCGTACTACCATCACCATC (SEQ ID NO: 15), downstream primer: cccaccggtAATATGAGCGTCACCTTTAGTTGC (SEQ ID NO: 16), the PCR program is \textcircled{1} 94° C. for 5 minutes, \textcircled{2} 94° C. for 30 seconds, \textcircled{3} 58° C. for 30 seconds, \textcircled{4} 72° C. for 1 minute, \textcircled{2} \textcircled{3} \textcircled{4} for 30 cycles, \textcircled{5} 72° C. for 5 minutes, and \textcircled{6} keeping at 4° C.). AgeI restriction sites were added at both its 5′ and 3′ ends. The synthesized SC cDNA fragment (1 μg) and pET21a-AP205 plasmid (1 μg) were digested with AgeI endonuclease (NEB R0552V) and ligated to construct pET21a-AP205-SC plasmid.

### (3) Construction of Fusion Protein AP205-ST (ST is Located at the C-Terminus of AP205 Sequence) Expression Plasmid

The fusion protein AP205-ST expression plasmid was constructed in the same way as in above (1), except that the ST encoding DNA sequence (gcccacatcgtgatggtggacgcctacaagccgacgaag) was synthesized through the following process (the encoded amino acid sequence is SEQ ID NO: 4):

### Artificially Synthesized Primers:

```
  (SEQ ID NO: 17)
F: ccggtggtagcggcgcccacatcgtgatggtggacgcctacaagccga
cgaaga
  (SEQ ID NO: 18)
R: ccggtcttcgtcggcttgtaggcgtccaccatcacgatgtgggcgccg
ctacca
```

The DNA sequence encoding ST was obtained by annealing PCR (5 μl 200 μM Primer-F, 5 μl 200 μM Primer-R, 2 μl 10× annealing buffer (100 mM Tris 8.0, 1 M NaCl, 10 mM EDTA), 8 μl dH₂O; the PCR program was set to be at 99° C. for 3 min, and the temperature was decreased by 0.5° C. every 30 seconds for 99-20° C., and finally kept at 4° C.). The pET21a-AP205 plasmid (1 μg) was digested with AgeI endonuclease (NEB R0552V) at 37° C. for 2 hours. The digested pET21a-AP205 plasmid fragment was then separated by agarose gel electrophoresis. The isolated pET21a-AP205 plasmid fragment was purified using a small amount of DNA product purification kit (Zhuangmeng Bio ZP201-3). The ST DNA fragment obtained by PCR was further subjected to DNA ligation reaction with the purified pET21a-AP205 plasmid fragment to construct a pET21a-AP205 plasmid containing the DNA fragment (called pET21a-AP205-ST plasmid). The ligase was T4 DNA ligase (Takara 2011A), the ligation buffer was T4 DNA Ligase Buffer (Takara 2011A), the ratio of pET21a-AP205 plasmid fragment to ST DNA fragment in the ligation reaction was about 1:3, and the total DNA was about 200 ng. Ligation was done at 22° C. for 2 hours. The pET21a-AP205-ST plasmid was transformed into the expression host as follows: 15 μl of the ligation reaction solution was added to 150 μl of XLI-Blue competent E. coli (full gold CD401-02) at 42° C. for 1 minute. 150 μl was pipetted onto an ampicillin-resistant LB plate and the plate was incubated at 37° C. for 14-16 hours. Plasmid DNA was extracted with a plasmid purification kit (Transgene EM101-02) and verified by enzyme digestion, confirming that the pET21a-AP205-ST plasmid was successfully constructed.

(4) Fusion Protein Expression and Self-Assembly into VLP, Purification of VLP

Fusion protein expression: the BL21 (DE3) competent E. coli (CD601-02) that has been verified by sequencing to be transformed with the previously constructed plasmid was used. A single clone was picked into ampicillin-resistant LB medium and subject to shaking overnight at 37° C., 220 rpm. On the second day, the culture was expanded, and the inducer IPTG (Yisheng Biology 10902ES08) at a final concentration of 0.1 mM was added to induce the expression of the fusion protein when the OD value in the logarithmic growth phase was 0.6-0.9, and the bacteria was harvested after 5 hours of induction.

Purification: the harvested E. coli (6000 rpm for 10 minutes) was centrifuged to obtain cell pellets. The pellet was resuspended in 20 mM Tris pH 7.5. The bacteria were sonicated to obtain the lysed supernatant. The supernatant was centrifuged twice (5000 rpm for 10 minutes, 20000 g for 30 minutes) to remove insoluble impurities such as cell debris, and then particle proteins were centrifuged through a 30% sucrose cushion (in a 12 ml centrifuge tube, 2 ml 30% sucrose was added at the bottom, and 10 ml lysate supernatant was added on it, 33000 rpm for 3.5 hours). The particle proteins were resuspended with 1 ml PBS (KCl 2.6 mM, KH₂PO₄ 1.47 mM, NaCl 136 mM, Na₂HPO₄·12H₂O 8 mM), and then the impurity proteins were separated from the target protein by cesium chloride density gradient centrifugation (in a 5 ml ultracentrifuge tube, 2 ml of 50% cesium chloride was added, 2 ml of 24% cesium chloride was added, and finally 1 ml of sample was added) (200000 g, 22 hours). Samples in layers were collected and a protein gel was run to confirm the location of the target fusion protein. The corresponding layer of protein was taken and dialyzed in PBS for storage.

### (5) Results

SC-AP205, AP205-SC and AP205-ST expression plasmids can express the corresponding fusion protein well.

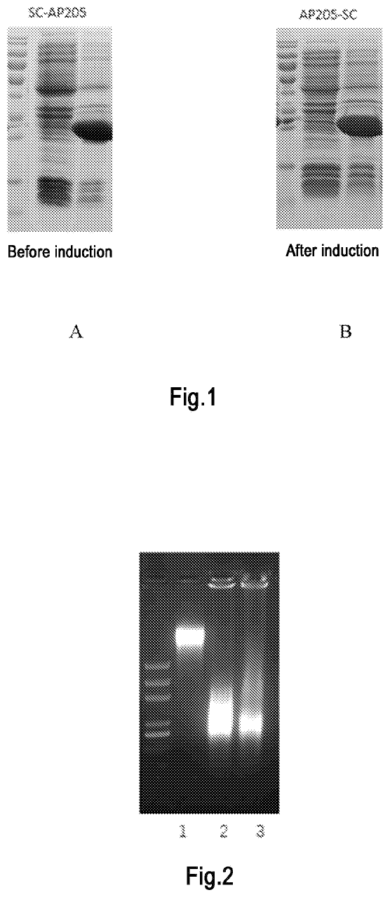

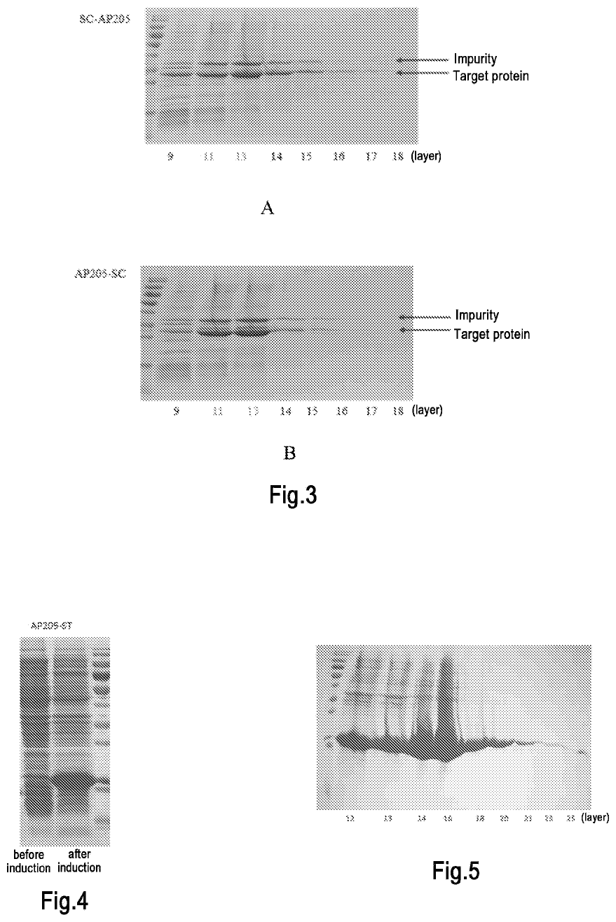

The comparison protein gel image of the whole bacterial lysate before and after induction of SC-AP205 and AP205-SC expression plasmids is shown in FIG. 1, showing that the target fusion protein was expressed. However, when purifying SC-AP205 and AP205-SC, the pellet at the bottom of the centrifuge tube was difficult to be resuspended after sucrose cushion centrifugation, indicating that there was a problem with the self-assembly of SC-AP205 and AP205-SC, and well-dispersed, non-aggregated VLPs could not be obtained (see FIG. 2). Samples in layers were collected after cesium chloride density gradient centrifugation. It was visibly turbid to the naked eye before the 13th layer, and it was clear after the 14th layer. However, from the protein gel band, the target protein could not be separated from the impurity protein, and the yield was very small (see FIG. 3).

For the AP205-ST expression plasmid, VLPs that were successfully self-assembled from AP205-ST were obtained in the subsequent purification (see FIGS. 2 and 4). Obvious target fusion protein bands could be seen in the 14th to 20th layers in the protein gel image of layered collection protein after the cesium chloride density gradient centrifugation (see FIG. 5). The corresponding layer was taken and dialyzed into PBS to obtain purified, well-dispersed, and non-aggregated VLPs assembled from AP205-ST (AP205-ST VLPs). In the laboratory stage, 50-60 mg VLP could be obtained per liter of bacteria.

### Example 2: The Effect of the Connecting Format of SpyCatcher (SC) and Antigen on the Stability of PLA

### (1) Construction of Fusion Protein RBD-SC (SC is Located at the C-Terminus of RBD) Expression Plasmid

The full-length 1068 bp cDNA (SEQ ID NO: 11) fragment encoding RBD-SC was artificially synthesized, and the Kozak sequence GCCACC for regulating protein expression and KpnI restriction site were added to its 5′ end and XhoI restriction site was added to the 3′ end. The synthesized RBD-SC cDNA fragment (1 μg) and pCEP4 plasmid (1 μg) were digested with KpnI and XhoI endonucleases (Takara) at 37° C. for 2 hours, respectively. The digested cDNA fragment and pCEP4 plasmid fragment were then separated by agarose gel electrophoresis. The isolated cDNA fragments and pCEP4 plasmid fragments were purified using a small amount of DNA product purification kit (Zhuangmeng Bio ZP201-3). The purified cDNA fragment was further subjected to DNA ligation reaction with the pCEP4 plasmid fragment to construct a pCEP4 plasmid containing the cDNA fragment (called pCEP4-RBD-SC plasmid). The ligase was T4 DNA ligase (Takara 2011A), the ligation buffer was T4 DNA Ligase Buffer (Takara 2011A), the ratio of the pCEP4 plasmid fragment to the RBD-SC cDNA fragment in the ligation reaction was about 1:3, and the total DNA was about 200 ng. Ligation was done at 22° C. for 2 hours. The pCEP4-RBD-SC plasmid was transformed into the expression host as follows: 15 μl of the ligation reaction solution was added to 150 μl of XLI-Blue competent E. coli (Transgene CD401-02) at 42° C. for 1 minute. 150 μl was pipetted onto an ampicillin-resistant LB plate and the plated was incubated at 37° C. for 14-16 hours. A single colony was taken from the plate, and the plasmid DNA was extracted with a plasmid purification kit (Transgene EM101-02) and verified by enzyme digestion, confirming the successful construction of the pCEP4-RBD-SC plasmid.

The pCEP4-RBD-SC plasmid was extracted from the host bacteria using the endotoxin-free large-scale extraction kit (Tiangen DP117). The extracted pCEP4-RBD-SC plasmid was transfected into 293F cell line (Life technologies) using PEI reagent (polyscience 23966-1). The transfection mixture was prepared: \textcircled{1} 300 μg of plasmid plus 15 ml of SMM 293-TII medium (Sino biological M293TII), \textcircled{2} 1.5 ml of PEI plus 15 ml of SMM 293-TII medium. The two were mixed and kept at room temperature for 2 minutes. The transfection mixture \textcircled{1} \textcircled{2}) were mixed fully and kept at room temperature for 15 minutes, followed by adding the transfection mixture to 300 ml of cell fluid with a cell density of 2×10⁵ cells/ml. They were mixed well and kept in a shaker at 37° C., 5% carbon dioxide, and 125 rpm. In the middle, 7 ml feed SMS 293-SUPI (Sino biological M293-SUPI) was added every two days, and the cells were harvested on the seventh day.

Insoluble impurities such as cell debris were removed by two-step centrifugation (500 g for 10 minutes, 8000 rpm for 30 minutes). The supernatant was passed through a 0.2 μm filter membrane to further remove insoluble impurities. Ni-NTA prepacked gravity column (BBI C600791-0005) was used to purify the expressed target protein. The steps are as follows:
a. Equilibration: it was first washed with 50 ml ultrapure water, and then 50 ml binding buffer (5 mM imidazole, 500 mM sodium chloride, 20 mM Tris, 10% glycerol, pH7.9) was used.
b. Sample loading: the cell supernatant was passed through the nickel column, and the sample loading was repeated for three times.
c. Elution: first, 50 ml of washing buffer (30 mM imidazole, 500 mM sodium chloride, 20 mM Tris, 10% glycerol, pH 7.9) was used to wash away impurities. The target protein was then eluted with 50 ml of elution buffer (250 mM imidazole, 500 mM sodium chloride, 20 mM Tris, 10% glycerol, pH 7.9). The target protein solution was concentrated to about 5 ml and then dialyzed into PBS for storage.

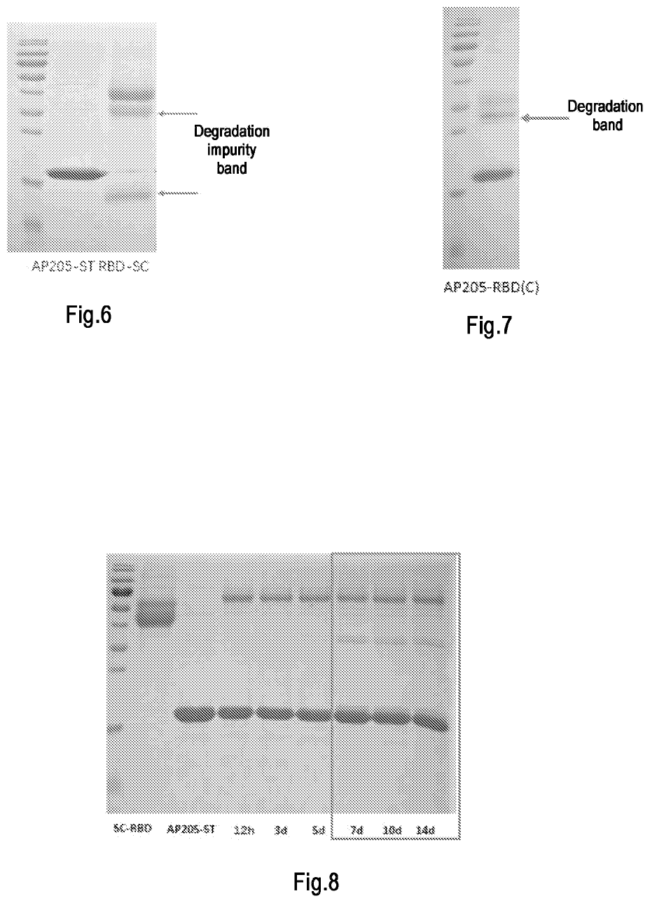

The obtained fusion protein RBD-SC had poor stability and was severely degraded when placed at 4° C. for three days (so the situation after three days was not shown in FIG. 6) (see FIG. 6).

### (2) Connection Between RBD-SC and AP205-ST VLP

RBD-SC and AP205-ST VLP at a ratio of 1:10 (one VLP is self-assembled from 180 AP205-ST sequences, i.e., there are 180 STs. This ratio refers to the ratio of the SC on the RBD and the ST on the VLP to be connected, the same below) was incubated in PBS buffer at 4° C. for 1 hour, whereby the Asp at the 7th position of the ST amino acid sequence and the Lys at the 31st position of the SC amino acid sequence spontaneously formed an isopeptide covalent bond, allowing RBD-SC to covalently connected to AP205-ST VLPs. This reaction process does not require any special enzymes and buffer systems.

The resulting connected product AP205-ST VLP/RBD-SC was completely degraded when left at 4° C. for 9 days (see FIG. 7).

### (3) Construction of Fusion Protein SC-RBD (SC is Located at the N-Terminus of RBD) Expression Plasmid and Connection of SC-RBD and AP205-ST VLP

The same method as above (1) was used to construct fusion protein SC-RBD expression plasmid, and the same method as above (2) was used to connect SC-RBD and AP205-ST VLP. The fusion protein SC-RBD sequence is SEQ ID NO: 8. The full-length 1059 bp SC-RBD cDNA (SEQ ID NO: 12) fragment was artificially synthesized, and the Kozak sequence GCCACC for regulating protein expression and HindIII restriction sites were added to its 5′ end and the XhoI restriction site was added to the 3′ end. The synthesized SC-RBD cDNA fragment (1 μg) and pCEP4 plasmid (1 μg) were digested with HindIII and XhoI endonucleases (Takara) at 37° C. for 2 hours, respectively.

Compared with the afore-mentioned fusion protein RBD-SC, the stability of the obtained fusion protein SC-RBD was obviously improved. Moreover, at 4° C., when the connection ratio of SC-RBD and AP205-ST VLP was 1:10, the connection product AP205-ST VLP/SC-RBD was stable within 5 days, and a small amount of antigen shedding did not occur until the 7th day. At the 14th days, most of them were still complete connected products (see FIG. 8). It could be seen that the stability of AP205-ST VLP/SC-RBD was significantly better than that of AP205-ST VLP/RBD-SC which was completely degraded on the 9th day under the same conditions.

### Example 3: Effect of the Sequence of AP205 on the Solubility of PLA

To study the effect of the sequence of AP205 on PLA solubility and the ability of AP205-ST VLP to carry external antigens, the inventors used the modified AP205 capsid protein sequence used in the present invention (i.e., five amino acids MEFGS were added to the N terminus of the wild-type (WT) AP205 capsid protein sequence. Unless otherwise specified, the AP205 used herein and the corresponding VLP and vaccine products were all prepared using the modified AP205) and the unmodified WT AP205 capsid protein sequence to perform a series of comparative experiments.

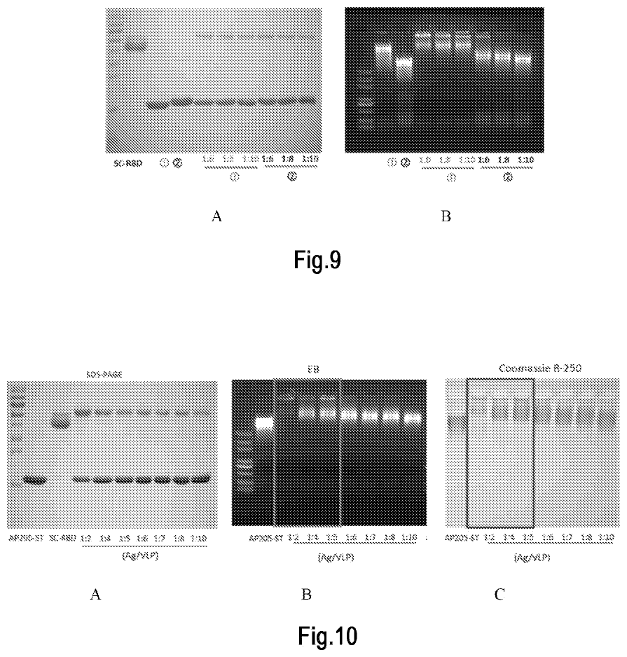

WT AP205-ST VLPs were obtained in the same manner as described above. Then it was connected with the fusion protein SC-RBD in the same way as the afore-mentioned connection method to obtain the corresponding connected product. The connected product was reduced and denatured and then subjected to SDS-PAGE to show the covalent connection between the antigen and VLP (FIG. 9A); and nucleic acid gel electrophoresis was run to detect the solubility of the connected product (FIG. 9B). The specific measurement process and conditions are as follows: the loading amount was 10 μg PLA or connection product, 1% nucleic acid gel, 90 volts, 20 minutes.

It was found that: SC-RBD could be well connected with wild-type and modified AP205-ST VLPs at different ratios (1:6, 1:8, 1:10) (see FIG. 9A). The connected products formed by WT AP205-ST VLPs were easy to aggregate, forming visible precipitates, which can be seen in the nucleic acid gel holes; while the solubility of the connected products formed by the modified AP205-ST VLPs was significantly improved, and there was no obvious visible precipitation, especially in 1:8 and 1:10 ratios (see FIG. 9B, in which \textcircled{1} was before modification and \textcircled{2} was after modification). Other antigens, including the African swine fever virus antigen eP22 (SEQ ID NO: 24), influenza virus antigen M2E (SEQ ID NO: 25) and autoantigen myelin oligodendrocyte glycoprotein MOG (SEQ ID NO: 26), were tested for the solubility of the connected products formed by connecting with the AP205-ST VLP before and after modification. The solubility of the connected products formed by the modified AP205-ST VLP was improved compared with the connected products formed by WT AP205-ST VLP.

### Example 4: Effect of the Ratio of Antigen to VLP on PLA Solubility

The present inventors studied the effect of the ratio of antigen to VLP on the solubility of connected product PLA, to further improve the solubility of the connected product by adjusting the ratio. The inventor used SC-RBD and AP205-ST VLP to test the ratios of 1:2, 1:4, 1:5, 1:6, 1:7, 1:8, and 1:10 respectively, and the test method was the same as that in Example 3. FIG. 10A shows that connected products could be successfully obtained under these ratios; FIG. 10B shows that when the ratio of antigen to VLP was higher (1:2, 1:4, 1:5), obvious deposition could be seen in the nucleic acid gel hole, and there was obvious precipitation visible to the naked eye, indicating that there was PLA aggregation at this time. However, when the connection ratio between antigen and VLP was reduced, for example, when the connection ratio was 1:6, 1:7, 1:8, and 1:10, there was basically no visible precipitation, and there was no protein deposited in the nucleic acid gel hole. FIG. 10C is the protein staining result of the agarose gel, which shows the movement of RNA and AP205 protein in the electrophoresis. It can also be seen that there was EB fluorescence and protein staining appeared in the gel hole when the connection ratio was high, indicating PLA aggregation. It could be seen that reducing the ratio of antigen to VLP can significantly improve the solubility of the connected product.

The inventor also used the same method to test the solubility of the connected products between AP205-ST VLP and the fusion protein of African swine fever virus antigen eP22 (SEQ ID NO: 24), influenza virus antigen M2E (SEQ ID NO: 25) and autoantigen myelin oligodendrocyte glycoprotein MOG (SEQ ID NO: 26) in SC-antigen format under different ratios. Combining SDS-PAGE protein gel, nucleic acid gel, and Coomassie R-250 protein staining results, it was found that:

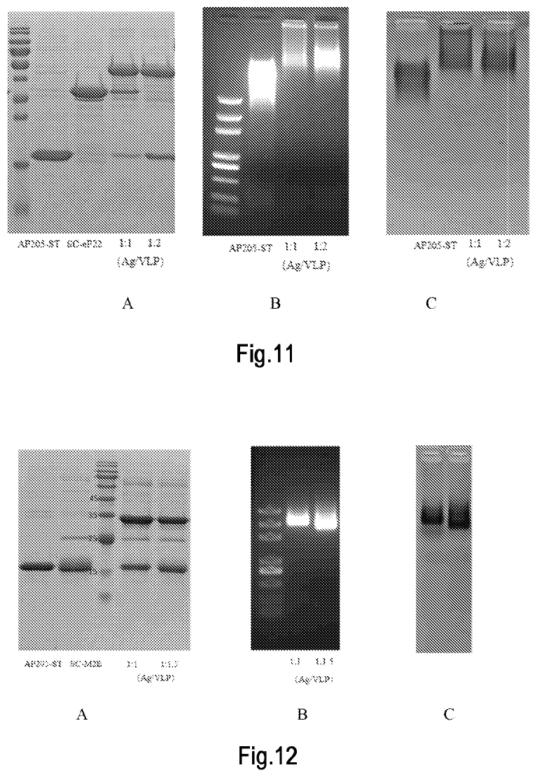

For the African swine fever virus antigen eP22, when the connection ratio is as high as 1:2, there is no visible deposition (see FIG. 11). Thus, the connection ratio suitable for the African swine fever virus antigen eP22 can be determined as 1:1˜1:5, such as 1:1, 1:2, 1:3, 1:4, 1:5.

For influenza virus antigen M2E, when the connection ratio reaches 1:1, there is no visible deposition (see FIG. 12). Thus, the connection ratio suitable for influenza virus antigen M2E can be determined as 1:1 to 1:1.5.

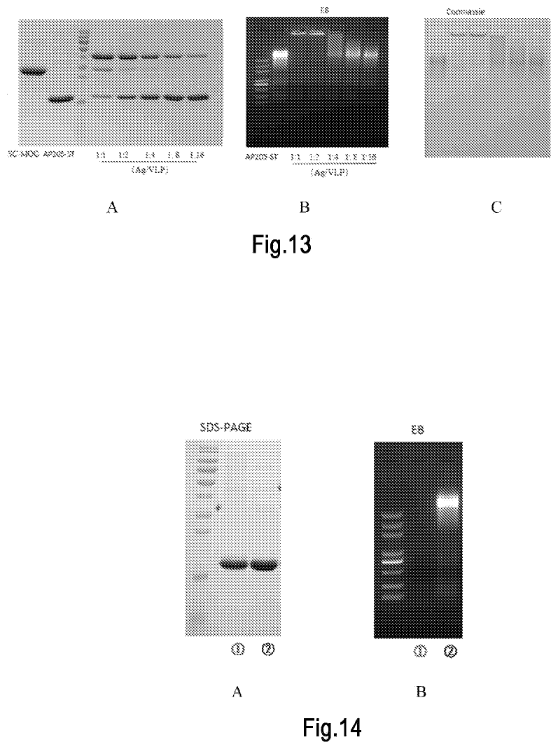

For autoantigen MOG, when the connection ratio reaches 1:4, there is no visible deposition of the connected product (see FIG. 13). Thus, the connection ratio suitable for autoantigen MOG can be determined as 1:4-1:10, such as 1:4, 1:5, 1:6, 1:7, 1:8, 1:10.

It could be seen that when different antigens in the SC-antigen format are connected to AP205-ST VLP, the connection ratio suitable for forming soluble connected product PLA is different. In other words, the type of antigen itself has an impact on the solubility of the connected product, but the trend is the same, i.e., as the connection ratio decreases, the solubility of the connected product gradually increases.

### Example 5: Effect of VLP Purification Conditions on the Presence or Absence of RNA Inside it

When exploring the industrial purification process of VLP, the inventors found that the RNA inside the purified VLP disappeared when the pH of the ion exchange solution was 10.5 (see FIG. 14), suggesting that the pH of the solution may affect the presence of RNA inside the VLP. Therefore, based on the VLP purification conditions described in Example 1, the inventors examined the effect of solution pH on the presence of RNA inside VLP. The specific method is as follows: adjusting the pH of PBS with hydrochloric acid and NaOH respectively, then placing 2.5 μg of purified VLP in a 37° C. water bath for 2 hours, and then detecting the effect of the pH of the solution on the presence of RNA inside the VLP by agarose gel electrophoresis and EB staining.

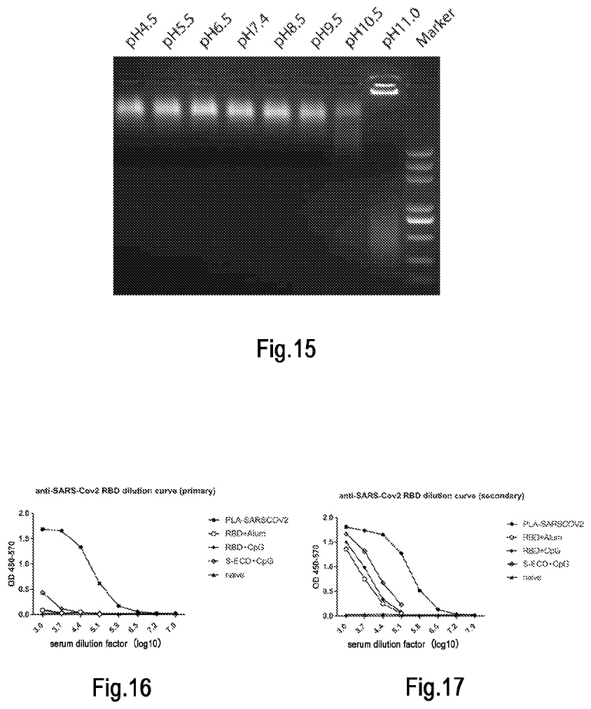

The results showed that the RNA content inside the VLP was stable within the pH range of 4.5-8.5 and began to decrease at pH 9.5. The RNA inside the VLP decreased significantly at pH 10.5 and above, and the RNA could not be detected at pH 11.0. The appearance of RNA outside VLP indicated that RNA would be released from inside the VLP under such alkaline conditions (see FIG. 15). The RNA inside the VLP of PLA plays a key role in the B cell-related immune activation mechanism of PLA (Sheng Hong et al., B Cells Are the Dominant Antigen-Presenting Cells that Activate Naive CD4+ T Cells upon Immunization with a Virus-Derived Nanoparticle Antigen, Immunity, 2018.10, 49:1-14). The test found that when there is RNA inside the VLP of PLA, the RNA acts as a TLR stimulator, enabling PLA to work by relying on B cell-related immune mechanisms, and the immune effect is better than that of PLA without RNA inside the VLP. Therefore, the inventor proposed that the purification process of VLP needs to be under suitable pH conditions, such as pH 4.0-9.0, and strong alkaline conditions above pH 10.5 should be avoided.

### Example 6: Ability of PLA-SARS-CoV2 Vaccine to Induce Anti-Novel Coronavirus RBD Antibodies

C57BL/6 mice (purchased from SPF) were divided into four groups: (1) RBD antigen mixed with aluminum adjuvant (Alum, purchased from Pierce), 12 mice, 10 μg/mouse; (2) RBD antigen mixed with CpG1826 adjuvant (sequence is tccatgacgttcctgacgtt), 4 mice, 10 μg/mouse (the amount of CpG is 50 μg/mouse); (3) extracellular segment of S protein mixed with aluminum adjuvant, 4 mice, 50 μg/mouse; (4) PLA-SARS-CoV2 (i.e., the vaccine complex formed by connecting the VLP formed after AP205 modification and the SARS CoV2 RBD antigen, the same below), 21 mice, 10 μg/mice. Intraperitoneal immunization was used. Blood was collected 14 days after the first immunization, which was recorded as the first immunization serum. The second immunization was carried out 21 days after the first immunization, and blood was collected 7 days after the second immunization (i.e., 28 days after the first immunization), which was recorded as the second immunization serum.

RBD-specific antibody response was detecte by ELISA. The coating amount of RBD antigen was 2 μg/ml, 50 μl/well, and kept overnight at 4° C. The serum was diluted in a gradient (the initial dilution of the serum was 1:1000, and then continued to be diluted 5 times, and a total of 8 gradients were made), followed by incubating with the RBD-coated Elisa 96-well plate at room temperature for 3 hours. Secondary antibody IgG-HRP (Bethyl Laboratories) was incubated at room temperature for 1 hour. After color development, the OD value of the corresponding well was read in a microplate reader. The wells without serum incubation were taken as the blank control. The average value of the OD values of 4-8 blank control wells plus 10 times the standard deviation is the reference value, and the lowest dilution of serum greater than the reference value is recorded as the antibody titer.

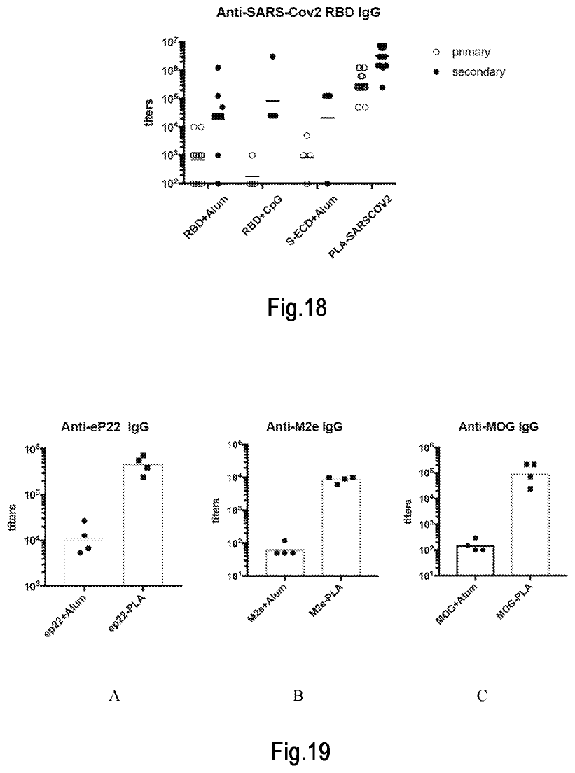

Regarding the specific measured values of different groups, the vertical axis is the OD reading value, and the abscissa axis is the Log value of serum dilution. It could be seen that the mice can produce a higher titer of RBD IgG antibody after one immunization (see FIG. 16). After the second immunization, the RBD IgG antibody titer can reach about 3×10⁶ (see FIG. 17). Compared with that after immunization with RBD antigen mixed with aluminum adjuvant, RBD antigen mixed with CpG adjuvant, and S protein extracellular segment of novel coronavirus mixed with aluminum adjuvant, the titer of the RBD IgG type antibody produced by PLA-SARS-CoV2 vaccine after the initial immunization or re-immunization could be increased by about 100 times (see FIG. 18).

The inventor further tested the antibody response of PLA vaccine constructed by influenza virus M2E antigen, African swine fever virus eP22 antigen and autoantigen MOG with the same method. The results showed that the PLA vaccine constructed by African swine fever virus eP22, influenza virus M2E antigen and autoantigen MOG could also cause a good antibody response. 14 days after immunizing C57BL/6 mice with several antigen based PLA vaccines, serum were collected to detect IgG type antibody response. It was found that compared with the corresponding antigen supplemented with adjuvant, PLA vaccines could induce good specific IgG antibody levels (see FIG. 19A, B, C, where each point in the figure represents the serum antibody titer level of a mouse).

### Example 7: Neutralizing Antibodies Produced in Mice Immunized with PLA-SARS-CoV2 Vaccine

Using RBD antigen mixed with aluminum adjuvant, RBD antigen mixed with CpG1826 adjuvant, extracellular segment of S protein mixed with aluminum adjuvant, and PLA-SARS-CoV2, induction of neutralizing antibody was compared by the following neutralizing antibody detection method as follows: serum dilution in 300 μl 2% DMEM medium, 3-fold dilution. 200 μl of serum at different dilutions were incubated with MOI 0.01 live virus (10 μl) at 37° C. for 1 hour. 200 μl was used to infect VERO-E6 cells in a 48-well plate. The medium was changed after 1 hour, and the cells were cultured in 2% DMEM medium for 24 hours. 150 μl supernatant was collected to extract RNA by MiniBEST Viral RNA/DNA Extraction Kit (Takara), and cDNA was reverse transcribed by PrimeScript™ RT reagent Kit with gDNA Eraser (Takara). The copy number was measured by the standard curve method (ABI 7500 (Takara TB Green® Premix Ex Taq II)) with the primers targeting the S gene.

(SEQ ID NO: 19)
Upstream primer (5′-3′): CAATGGTTTAACAGGCACAGG;
(SEQ ID NO: 20)
Downstream primer (5′-3′): CTCAAGTGTCTGTGGATCACG.

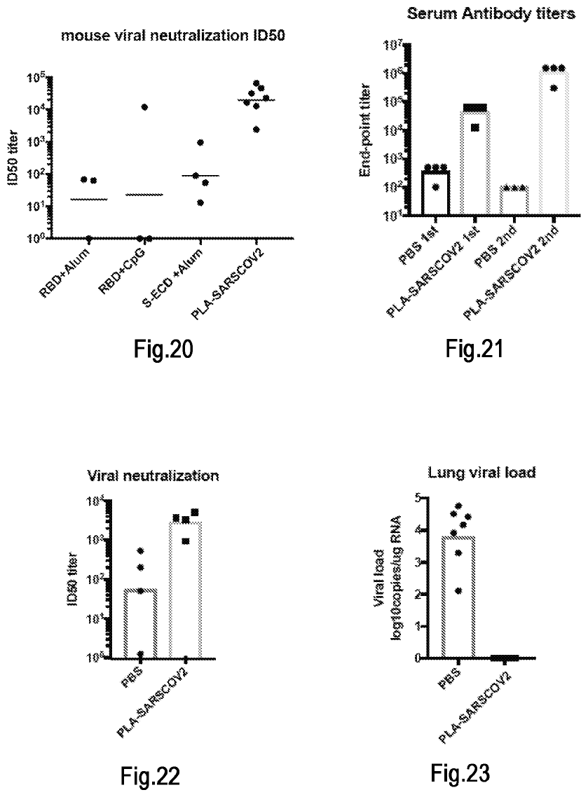

The vertical axis is the neutralizing antibody titer (ID50 titer) detected by ELISA, which shows that the level of neutralizing antibody induced by PLA-SARS-CoV2 of the present invention is 100 times higher than that of other traditional vaccines mixed with adjuvants (see FIG. 20).

### Example 8: Anti-RBD IgG Antibodies Produced in Macaques Immunized with PLA-SARS-CoV2 Vaccine

Eight young healthy macaques (male, 3-6 years old, all from Kunming Primate Research Center, Chinese Academy of Sciences) were used in the experiment. The immunization test was divided into two groups (4 for each) and each received intramuscular injection of PLA-SARS-CoV2 (20 μg/macaque/time) or normal saline (PBS, control group) twice (interval of 3 weeks). Blood was collected 14 days after the first injection and 7 days after the second injection, followed by serum separation. For the method of detecting anti-RBD IgG antibody titer, refer to the ELISA method described in Example 6. The secondary antibody was replaced with HRR-labeled goat anti-monkey IgG (purchased from Abcam, Cat. No. ab112767). FIG. 21 shows the anti-RBD IgG antibody levels in the serum 14 days after the initial immunization (1st) and 7 days after the second immunization (2nd) (the vertical axis is the antibody titer detected by ELISA), indicating that the anti-RBD IgG antibody level in the serum at the time of initial immunization was more than 100 times that of the PBS control, and the anti-RBD IgG antibody level after re-immunization was more than 1000 times higher than that of the PBS control.

### Example 9: Neutralizing Antibodies Produced in Macaques Immunized with PLA-SARS-CoV2 Vaccine

The inventors further tested the neutralizing antibodies induced by PLA-SARS-CoV2 vaccine of the present invention in macaques. The immunization process and conditions were the same as those in Example 8 above. The method of detecting serum neutralizing antibody titer was the same as that in Example 7. FIG. 22 shows that the level of neutralizing antibodies to the novel coronavirus in the serum after 7 days of re-immunization (2nd) increased dozens of times relative to the PBS control.

### Example 10: Viral Load in the Lung of Macaques Immunized with PLA-SARS-CoV2 Vaccine

The inventors next tested the viral load in the lung of macaques immunized with the PLA-SARS-CoV2 vaccine. The process of immunizing macaques with PLA-SARS-CoV2 vaccine was the same as that in Example 8. The virus used in the experiment was Novel Coronavirus 107 strains (provided by the Guangdong Provincial Center for Disease Control and Prevention in China). The virus strains were expanded by Vero-E6 cell lines, and half of the tissue culture infection dose was determined by the Reed-Muench method.

Viral challenge experiments were performed 10 days after re-immunization. The way of challenge is a combination method, intranasal (0.4 mL/nostril) and intratracheal (1.2 mL, fiberoptic bronchoscope). The total virus titer was 1×10⁷ TCID50 mL, which was diluted with sterile 0.9% normal saline.

The viral load in lung of the two groups of animals after 7 days was detected by RT-PCR. Total RNA was extracted from swabs and tracheal brushes using a kit (Roche Germany), and RNA from tissue samples was extracted using TRIzol reagent (Thermo USA). Viral RNA was detected using a probe-one-step real-time quantitative PCR kit (TOYOBO, Japan). The primers and probe are as follows: upstream primer 5′-GGGGAACTTCTCCTGCTAGAAT-3′ (SEQ ID NO: 21), downstream primer 5′-CAGACATTTTGCTCTCCAAGCTG-3′ (SEQ ID NO: 22) and FAM-TTGCTGCTGCTTGACAGATT-TAMRA-3′ (SEQ ID NO: 23). Each test sample was diluted according to the standards of the National Institute of Metrology of China, and finally the copy number of each sample was calculated.

The results showed that the virus in the lung of the macaque almost completely disappeared after immunization with the PLA-SARS-CoV2 vaccine of the present invention (FIG. 23, the vertical axis of the figure shows the logarithmic value of the virus copy number per μg RNA). Moreover, according to the results of induced specific antibodies and neutralizing antibodies by the PLA-SARS-CoV2 vaccine of the present invention in the foregoing examples, it can be reasonably inferred that the vaccine of the present invention would have significantly better results in the challenge test compared to other traditional vaccines that require additional adjuvants.

1. A soluble pathogen-like antigen (PLA) complex comprising:
(1) a virus-like particle (VLP), which is self-assembled by a first fusion protein comprising a viral capsid protein or a variant thereof at its N-terminus, SpyTag at its C-terminus, and a first linker peptide linking both;
(2) a second fusion protein, comprising an antigen or a variant thereof, SpyCatcher and a second linker peptide linking both, preferably the SpyCatcher is at the N-terminus of the second fusion protein;
wherein the virus-like particle also encapsulates nucleic acid inside it, and
wherein the virus-like particle and the second fusion protein are covalently connected through the SpyCatcher and the SpyTag, so that the antigen or a variant thereof is displayed on the surface of the virus-like particle.

2. The soluble pathogen-like antigen complex according to claim 1, wherein the nucleic acid encapsulated in the virus-like particle is the nucleic acid which is encapsulated by the virus-like particle when self-assembling and is from host bacteria expressing the virus-like particles, preferably the host bacteria is Escherichia coli, and preferably the nucleic acid is RNA.

3. The soluble pathogen-like antigen complex according to claim 1, wherein the capsid protein is from Escherichia coli phage Qβ, MS2 or AP205, preferably from Escherichia coli phage AP205.

4. The soluble pathogen-like antigen complex according to claim 1, wherein the antigen is selected from RBD sequence of S protein of SARS-CoV2 virus, African swine fever virus antigen eP22, influenza virus antigen M2E and autoantigen myelin oligodendrocyte glycoprotein MOG.

5. The soluble pathogen-like antigen complex according to claim 3, wherein the sequence of the capsid protein of the phage AP205 has at least 90%, 91%, 92%, 93%, 94%, 95%, 96%, 97%, 98%, 99% or 100% identity with SEQ ID NO: 1.

6. The soluble pathogen-like antigen complex according to claim 1, wherein the sequence of the SpyTag has at least 90%, 91%, 92%, 93%, 94%, 95%, 96%, 97%, 98%, 99% or 100% identity with of SEQ ID NO: 3.

7. The soluble pathogen-like antigen complex according to claim 1, wherein the sequence of the SpyCatcher has at least 90%, 91%, 92%, 93%, 94%, 95%, 96%, 97%, 98%, 99% or 100% identity with SEQ ID NO: 4.

8. The soluble pathogen-like antigen complex according to claim 1, wherein:
(1) the SpyTag has at least 90%, 91%, 92%, 93%, 94%, 95%, 96%, 97%, 98%, 99% or 100% sequence identity to SEQ ID NO: 3;
(2) the SpyCatcher has at least 90%, 91%, 92%, 93%, 94%, 95%, 96%, 97%, 98%, 99% or 100% sequence identity to SEQ ID NO: 4; and
(3) an isopeptide bond is formed between Asp at position 7 of the SpyTag sequence SEQ ID NO: 3 and Lys at position 31 of the SpyCatcher sequence SEQ ID NO: 4.

9. The soluble pathogen-like antigen complex according to claim 1, wherein the sequence of the first linker peptide is SEQ ID NO: 5.

10. The soluble pathogen-like antigen complex according to claim 1, wherein the sequence of the second linker peptide is SEQ ID NO: 6.

11. The soluble pathogen-like antigen complex according to claim 1, wherein the second fusion protein is connected to the virus-like particle at a ratio of less than or equal to 1:1 according to different antigens antigents, preferably connected at a ratio of 1:1, 1:1.5, 1:2, 1:3, 1:4, 1:5, 1:6, 1:7, 1:8, 1:9, 1:10, 1:11, or 1:12, so as to ensure the solubility and immunogenicity of the pathogen-like antigen complex, wherein the ratio is calculated as the ratio of the SpyCatcher on the second fusion protein to the SpyTag on the virus-like particle.

12. A pathogen-like antigen vaccine composition, comprising the soluble pathogen-like antigen complex according to claim 1 and pharmaceutically acceptable carriers and/or excipients.

13. A method for preparing the soluble pathogen-like antigen complex of claim 1, comprising purifying the virus-like particles at a pH range of 4.0 to 9.0, preferably 5.5 to 8.5.

14. A method for improving the solubility of a pathogen-like antigen complex, comprising:
(1) preparing the virus-like particle and the second fusion protein of claim 1; and
(2) when connecting the second fusion protein with the virus-like particle, reducing a connection ratio between the second fusion protein and the virus-like particle to obtain a soluble pathogen-like antigen complex.

15. A method for preventing and/or treating diseases associated with SARS-CoV2 virus, influenza virus, African swine fever virus or autoantigen myelin oligodendrocyte glycoprotein MOG in a subject in need thereof, comprising administering a preventive and/or therapeutically effective amount of the pathogen-like antigen vaccine composition of claim 12 to the subject.


SEQUENCE LISTING (26 sequences)

SEQ ID NO: 1 - protein, 136 aa, Artificial Sequence
  AP205 amino acid sequence
MEFGSMANKPMQPITSTANKIVWSDPTRLSTTFSASLLRQRVKVGIAELNNVSGQYVSVYKRPAPKPEGCADACVIMPNENQSIRTVISGSAENLATLKAEWETHKRNVDTLFASGNAGLGFLDPTAAIVSSDTTA

SEQ ID NO: 2 - protein, 223 aa, Artificial Sequence
  Covid-19 RBD amino acid sequence
RVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKSVNF

SEQ ID NO: 3 - protein, 15 aa, Artificial Sequence
  ST amino acid sequence
AHIVMVDAYKPTKTG

SEQ ID NO: 4 - protein, 92 aa, Artificial Sequence
  SC amino acid sequence
DSATHIKFSKRDEDGKELAGATMELRDSSGKTISTWISDGQVKDFYLYPGKYTFVETAAPDGYEVATAITFTVNEQGQVTVNGKATKGDAHI

SEQ ID NO: 5 - protein, 11 aa, Artificial Sequence
  first linker peptide amino acid sequence
GSGGSGTGGSG

SEQ ID NO: 6 - protein, 6 aa, Artificial Sequence
  second linker peptide amino acid sequence
GSGGSG

SEQ ID NO: 7 - protein, 162 aa, Artificial Sequence
  AP205-first linker peptide-ST amino acid sequence
MEFGSMANKPMQPITSTANKIVWSDPTRLSTTFSASLLRQRVKVGIAELNNVSGQYVSVYKRPAPKPEGCADACVIMPNENQSIRTVISGSAENLATLKAEWETHKRNVDTLFASGNAGLGFLDPTAAIVSSDTTAGSGGSGTGGSGAHIVMVDAYKPTKTG

SEQ ID NO: 8 - protein, 321 aa, Artificial Sequence
  SC-second linker peptide-RBD amino acid sequence
DSATHIKFSKRDEDGKELAGATMELRDSSGKTISTWISDGQVKDFYLYPGKYTFVETAAPDGYEVATAITFTVNEQGQVTVNGKATKGDAHIGSGGSGRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKSVNF

SEQ ID NO: 9 - DNA, 393 bp, Artificial Sequence
  AP205 cDNA sequence
atggcaaataagccaatgcaaccgatcacatctacagcaaataaaattgtgtggagtgatccaactcgtttatcaactacattttcagcaagtctgttacgccaacgtgttaaagttggtatagccgaactgaataatgtttcaggtcaatatgtatctgtttataagcgtcctgcacctaaaccggaaggttgtgcagatgcctgtgtcattatgccgaatgaaaaccaatccattcgcacagtgatttcagggtcagccgaaaacttggctaccttaaaagcagaatgggaaactcacaaacgtaacgttgacacactcttcgcgagcggcaacgccggtttgggtttccttgaccctactgcggctatcgtatcgtctgatactactgct

SEQ ID NO: 10 - DNA, 276 bp, Artificial Sequence
  SC cDNA sequence
gatagcgctacccatatcaaattctcaaaacgtgatgaggacggcaaagagttagctggtgcaactatggagttgcgtgattcatctggtaaaactattagtacatggatttcagatggacaagtgaaagatttctacctgtatccaggaaaatatacatttgtcgaaaccgcagcaccagacggttatgaggtagcaactgctattacctttacagttaatgagcaaggtcaggttactgtaaatggcaaagcaactaaaggtgacgctcatatt

SEQ ID NO: 11 - DNA, 1068 bp, Artificial Sequence
  RBD-SC cDNA sequence
atgttcgtgtttctggtgctgctgcctctggtgagctcccagtgcaccggtagggtgcagcctaccgagtctatcgtgcgcttcccaaacatcacaaatctgtgccccttcggcgaggtgtttaatgccacccggtttgccagcgtgtatgcctggaatcggaagagaatctccaactgcgtggccgactattctgtgctgtacaacagcgccagcttcagcaccttcaagtgctatggcgtgtcccccaccaagctgaatgacctgtgcttcacaaacgtgtacgccgactcttttgtgatccggggcgatgaggtgagacagatcgcaccaggacagaccggcaagatcgcagactacaattataagctgcctgacgatttcacaggctgcgtgatcgcctggaacagcaacaatctggattccaaagtgggcggcaactacaattatctgtacaggctgttccgcaagtccaatctgaagccatttgagagggacatcagcaccgagatctaccaggccggctccacaccctgcaatggcgtggagggcttcaactgttattttcctctgcagagctacggcttccagccaaccaacggcgtgggctatcagccctaccgcgtggtggtgctgtcctttgagctgctgcacgcaccagcaaccgtgtgcggacctaagaagtctacaaacctggtgaagaataagagcgtgaactttggaggaggaggaggaggaagcaagcttgactctgccacccacatcaagttcagcaagagggacgaggatggcaaggagctggcaggagcaacaatggagctgagagatagctccggcaagaccatctccacatggatctctgacggccaggtgaaggatttctacctgtatcctggcaagtatacctttgtggagacagcagcaccagacggatacgaggtggcaaccgccatcacctttacagtgaatgagcagggccaggtgaccgtgaacggcaaggccacaaagggcgatgcacacatcggaggaagccatcatcatcatcatcaccatcatcatcatcatcac

SEQ ID NO: 12 - DNA, 1059 bp, Artificial Sequence
  SC-RBD cDNA sequence
atgttcgtgtttctggtgctgctgcctctggtgagctcccagtgcgactctgccacccacatcaagttcagcaagagggacgaggatggcaaggagctggcaggagcaacaatggagctgagagatagctccggcaagaccatctccacatggatctctgacggccaggtgaaggatttctacctgtatcctggcaagtatacctttgtggagacagcagcaccagacggatacgaggtggcaaccgccatcacctttacagtgaatgagcagggccaggtgaccgtgaacggcaaggccacaaagggcgatgcacacatcggatctggaggaagcggaagggtgcagcctaccgagtctatcgtgcgcttcccaaacatcacaaatctgtgccccttcggcgaggtgtttaatgccacccggtttgccagcgtgtatgcctggaatcggaagagaatctccaactgcgtggccgactattctgtgctgtacaacagcgccagcttcagcaccttcaagtgctatggcgtgtcccccaccaagctgaatgacctgtgcttcacaaacgtgtacgccgactcttttgtgatccggggcgatgaggtgagacagatcgcaccaggacagaccggcaagatcgcagactacaattataagctgcctgacgatttcacaggctgcgtgatcgcctggaacagcaacaatctggattccaaagtgggcggcaactacaattatctgtacaggctgttccgcaagtccaatctgaagccatttgagagggacatcagcaccgagatctaccaggccggctccacaccctgcaatggcgtggagggcttcaactgttattttcctctgcagagctacggcttccagccaaccaacggcgtgggctatcagccctaccgcgtggtggtgctgtcctttgagctgctgcacgcaccagcaaccgtgtgcggacctaagaagtctacaaacctggtgaagaataagagcgtgaactttggaggaggaggaagccatcatcatcatcatcaccatcatcatcatcatcac

SEQ ID NO: 13 - DNA, 31 bp, Artificial Sequence
  SC-AP205 expression vector upstream primer sequence
acgggatccatgtcgtactaccatcaccatc

SEQ ID NO: 14 - DNA, 48 bp, Artificial Sequence
  SC-AP205 expression vector downstream primer sequence
cccggatccactgccgctacctccaatatgagcgtcacctttagttgc

SEQ ID NO: 15 - DNA, 31 bp, Artificial Sequence
  AP205-SC expression vector upstream primer sequence
acgaccggtatgtcgtactaccatcaccatc

SEQ ID NO: 16 - DNA, 33 bp, Artificial Sequence
  AP205-SC expression vector downstream primer sequence
cccaccggtaatatgagcgtcacctttagttgc

SEQ ID NO: 17 - DNA, 54 bp, Artificial Sequence
  AP205-ST expression vector upstream primer sequence
ccggtggtagcggcgcccacatcgtgatggtggacgcctacaagccgacgaaga

SEQ ID NO: 18 - DNA, 54 bp, Artificial Sequence
  AP205-ST expression vector downstream primer sequence
ccggtcttcgtcggcttgtaggcgtccaccatcacgatgtgggcgccgctacca

SEQ ID NO: 19 - DNA, 21 bp, Artificial Sequence
  the upstream primer sequence in Example 7
caatggtttaacaggcacagg

SEQ ID NO: 20 - DNA, 21 bp, Artificial Sequence
  the downstream primer sequence in Example 7
ctcaagtgtctgtggatcacg

SEQ ID NO: 21 - DNA, 22 bp, Artificial Sequence
  the upstream primer sequence in Example 10
ggggaacttctcctgctagaat

SEQ ID NO: 22 - DNA, 22 bp, Artificial Sequence
  the downstream primer sequence in Example 10
cagacattttgctctcaagctg

SEQ ID NO: 23 - DNA, 20 bp, Artificial Sequence
  the probe sequence in Example 10
ttgctgctgcttgacagatt

SEQ ID NO: 24 - protein, 148 aa, Artificial Sequence
  the African swine fever virus antigen eP22 amino acid sequence
KKQQPPKKVCKVDKDCGSGEHCVRGSCSSLSCLDAVKMDKRNIKIDSKISSCEFTPNFYRFTDTAADEQQEFGKTRHPIKITPSPSESHSPQEVCEKYCSWGTDDCTGWEYVGDEKEGTCYVYNNPHHPVLKYGKDHIIALPRNHKHA

SEQ ID NO: 25 - protein, 23 aa, Artificial Sequence
  influenza virus antigen M2E amino acid sequence
SLLTEVETPIRNEWGCRCNGSSD

SEQ ID NO: 26 - protein, 117 aa, Artificial Sequence
  autoantigen myelin oligodendrocyte glycoprotein MOG amino acid sequence
GQFRVIGPGYPIRALVGDEAELPCRISPGKNATGMEVGWYRSPFSRVVHLYRNGKDQDAEQAPEYRGRTELLKETISEGKVTLRIQNVRFSDEGGYTCFFRDHSYQEEAAMELKVED